(19) United States
(12) Patent Application Publication
(10) Pub. No.: US 2008/0161828 A1
(43) Pub. Date: Jul. 3, 2008

(54) Bone breaking instrument

(76) Inventor: Jason M. Yamada, Rolling Hills Estates, CA (US)

Correspondence Address: Robert R. Meads, 214 Paseo de Suenos, Redondo Beach, CA, 90277, US

(21) Appl. No.: 11/895,810

(22) Filed: Aug. 28, 2007

Related U.S. Application Data
(60) Provisional application No. 60/882,940, filed on Dec. 31, 2006.

(51) Int. Cl. A61B 17/22

(52) U.S. Cl. 606/128; 606/127

(57) ABSTRACT

A sinus bone-breaking instrument comprising a longitudinally extending handle and a relatively small laterally extending distal head designed to fit upward into a bone channel and having a laterally inward and upward inclined lower surface shaped to hook bone fragments in the bone channel and with a downward pulling on the handle to break off the bone fragments leaving the bone channel free of bone fragments.

### RELATED PATENT APPLICATIONS

The present application claims the benefit of U.S. Provisional Patent application Ser. No. 60/882,940 filed Dec. 31, 2006, which is herein incorporated by reference. The present application also relates to the subject matter of the concurrently filed U.S. patent application Ser. No. ______, entitled “Internal Sinus Manipulation (ISM) Procedure For Facilitating Sinus Floor Augmentation In Dental Procedures” (Docket 07JY-1), which is incorporated herein by this reference.

### BACKGROUND OF INVENTION

As stated in the above-identified concurrently filed United States patent application, during the described procedure and in the formation of an upward channel in the bone leading to the sinus floor of a patient, small inwardly directed bone fragments may surround or extend into the upper opening of the sinus bone channel. As illustrated in FIG. 4 of the above-identified concurrently filed United States patent application, after the formation of a small pocket between he floor of the sinus and the sinus membrane, such bone fragments are removed, preferably using a bone breaking instrument. The present invention is directed to a preferred form of such a sinus bone-breaking instrument.

### SUMMARY OF INVENTION

Basically, the sinus bone-breaking instrument of the present invention comprises a longitudinally extending handle and a relatively small laterally extending distal head designed to fit upward into a bone channel. To enable the distal head to effectively remove bone fragments extending into the bone channel, the distal head includes a laterally inward and upward inclined lower surface. The lower inclined surface is employed to hook the bone fragments and with a downward pulling on the handle to break off the bone fragments leaving a clear opening to the sinus membrane.

### BRIEF DESCRIPTION OF ACCOMPANYING DRAWINGS

FIG. 1 is a perspective view of the preferred bone-breaking instrument.

FIG. 2 is an enlarged fragmentary sectional side view of an upper portion of the instrument of FIG. 1 showing the laterally extending distal head of the instrument having an inwardly and upwardly inclined lower bone-hooking surface.

FIG. 3 is a slightly enlarged fragmentary sectional front view of the distal head of the instrument depicted in FIG. 2.

### DETAILED DESCRIPTION OF INVENTION

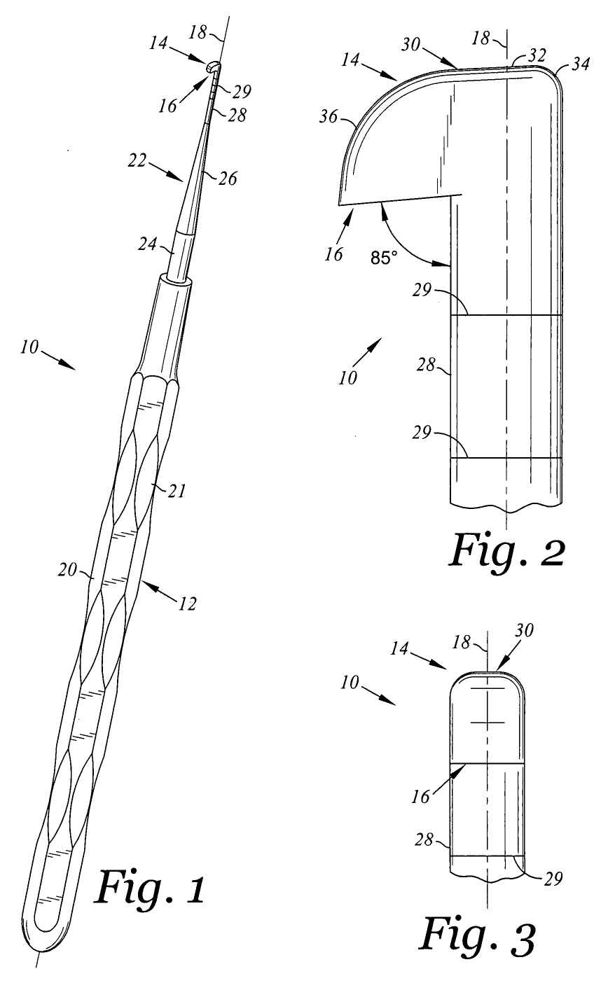

As depicted in FIG. 1, the bone-breaking instrument 10 of the present invention comprises a longitudinally extending handle 12 and a laterally extending distal head 14 with an inwardly and upwardly inclined lower surface 16.

As shown in FIG. 1, the handle 12 is longitudinally elongated on a longitudinal axis 18 of the instrument 10 and includes a slightly enlarged lower hand-holdable portion 20 having a longitudinally fluted outer surface 21 for enhancing the gripability of the instrument 10.

As illustrated, the distal head 14 is secured to the handle by an intermediate longitudinally extending portion 22 of the instrument 10 comprising a cylindrical lower section 24, an axially elongated frusto-conical middle section 26 and an upper cylindrical section 28 of reduced diameter compared to the diameter of the lower section 24. As depicted in FIG. 1, the cylindrical lower section 24 is secured to and is of a slightly reduced diameter relative an upper end of the hand-holdable portion 20 of the handle 12. The bottom of the frusto-conical middle section 26 is secured to a top of the lower section 24, is axially elongated on the axis 18 and is reduced in lateral diameter as to approaches its relatively small upper end joined to a lower end of the upper cylindrical section 28. The upper cylindrical section 28, in turn, extends, longitudinally on the axis 18 upward to support the distal head 14 that is secured to and supported by the end of the cylindrical section 28, as best shown in FIG. 2. As shown in FIGS. 1 and 2, the outer surface of the upper cylindrical section 28 below the distal head 14 carries a plurality of horizontal vertically and evenly spaced depth markers 29 for indicating the user of the instrument 10 the depth of the instrument in a bone channel prior to use in the breaking of bone fragments. By way of example, the vertical spacing of the depth markers 29 may be about 2 millimeters.

As most clearly shown in FIGS. 2 and 3, the distal head 14 extends upwardly from the upper end of the upper cylindrical section 28 and laterally from the longitudinal axis 18 of the instrument 10. An upper surface 30 of the distal head 14 is substantially flat and perpendicular to the axis 18 of the instrument 10 in its central region 32 and curved downwardly at its rearmost end 34 and at its forwardmost end 36 terminating at the lower surface 16. As illustrated in FIG. 2, the lower surface 16 is substantially flat and laterally and upwardly inclined toward the longitudinal axis 18 at an angle of less than 90 degrees to the longitudinal axis, preferably about 85 degrees as indicated. As illustrated in FIG. 3, the upper surface 30 of the distal head 14 is also curved downwardly at right and left side of the head 14 (as depicted in FIG. 3).

Thus configured, the instrument 10 is shaped for comfortable hand gripping with the handle 12, intermediate 22 and distal head 14 portions extending upwardly and into a bone channel to break off bone fragments extending inwardly from the wall of the channel. The lower inclined surface 16 of the distal head 14 is employed to hook the bone fragments and with a downward pulling on the handle 12 to break off the bone fragments leaving a clear opening in the bone channel to the sinus membrane.

While a particular preferred embodiment of the bone-breaking instrument has been illustrated and described above, it is appreciated that changes and modifications may be made in the illustrated embodiment without departing from the spirit of the invention. Accordingly, the scope of present invention is to be limited only by the terms of the following claims.

1. A sinus bone breaking tool, comprising:
a longitudinally extending handle and
a relatively small laterally extending distal head dimensioned to fit upward into a bone channel and including a laterally inward and upward inclined lower surface shaped to hook bone fragments in a bone channel and with a downward pulling on the handle to break off the bone fragments leaving a clear opening in the bone channel.

2. The instrument of claim 1 wherein:
the handle is longitudinally elongated on a longitudinal axis of the instrument and includes an enlarged lower hand-holdable portion having a longitudinally fluted outer surface for enhancing the gripability of the instrument.

3. The instrument of claim 1 wherein:
the distal head is secured to the handle by an intermediate longitudinally extending portion of the instrument comprising a cylindrical lower section, an axially elongated frusto-conical middle section and an upper cylindrical section of reduced diameter compared to the diameter of the lower section,
the cylindrical lower section being secured to and being of a slightly reduced diameter relative an upper end of the hand-holdable portion of the handle,
a bottom of the frusto-conical middle section being secured to a top of the lower section, axially elongated on the axis of the instrument and reduced in lateral diameter as to approaches a relatively small upper end joined to a lower end of the upper cylindrical section, and
the upper cylindrical section extending longitudinally on the axis of the instrument upward to support the distal head secured to and supported by the end of the cylindrical section.

4. The instrument of claim 1 wherein:
the distal head is secured to the handle by an intermediate longitudinally extending portion of the instrument and
the lower surface of the distal head is laterally and upwardly inclined toward the longitudinal axis at an angle of less than 90 degrees to the longitudinal axis.

5. The instrument of claim 4 wherein:
the inclined lower surface of the distal head extends upwardly from the intermediate portion and laterally from the longitudinal axis of the instrument.

6. The instrument of claim 5 wherein:
the inclined lower surface of the distal head extends upwardly from the intermediate portion and laterally from the longitudinal at an angle of less than 90 degrees to the axis of the instrument.

7. The instrument of claim 6 wherein the angle is about 85 degrees.

8. The instrument of claim 4 wherein:
an upper surface the distal head is substantially flat and substantially perpendicular to the axis of the instrument in a central region and curved downwardly at a rearmost end and at a forwardmost end terminating at the lower surface.

9. The instrument of claim 8 wherein:
the lower surface is substantially flat and laterally and upwardly inclined toward the longitudinal axis of the instrument at an angle of less than 90 degrees to the longitudinal axis.

10. The instrument of claim 9 wherein:
the angle is about 85 degrees.

11. The instrument of claim 8 wherein:
the upper surface of the distal head is also curved downwardly at right and left sides of the head.
